(19) United States
(12) Patent Application Publication
(10) Pub. No.: US 2005/0037132 A1
(43) Pub. Date: Feb. 17, 2005

(54) Anti-thrombogenic surfaces and process for their production

(76) Inventors: Roland Horres, Stolberg (DE); Michael Hoffmann, Eschweiler (DE); Sabine Kuesters, Niederzier (DE); Marita Katharina Linssen, Aachen (DE)

Correspondence Address: Leon R. Yankwich;Yankwich & Associates, 201 Broadway, Cambridge, MA, 02139, US

(21) Appl. No.: 10/422,152

(22) Filed: Apr. 24, 2003

Related U.S. Application Data
(60) Provisional application No. 60/375,002, filed on Apr. 25, 2002.

(51) Int. Cl. A61L 2/00

(52) U.S. Cl. 427/2.1

(57) ABSTRACT

The invention relates to anti-thrombogenic surfaces and a process for making said surfaces. The process is primarily used for the anti-thrombogenic coating of hydrophobic surfaces of medical products coming into direct contact with blood and coagulable blood products.

The present application claims priority to U.S. provisional application No. 60/375,002 filed Apr. 25, 2002, under 34 U.S. Code 119(e).

### FIELD OF THE INVENTION

The invention relates to anti-thrombogenic surfaces and a process for applying an anti-thrombogenic coating onto surfaces of materials as desired. The process is primarily used for the anti-thrombogenic coating of surfaces of medical products coming into direct contact with blood and blood products.

These medical products include, for example, hypodermic needles, tubes, syringes, needles and other objects capable of coming into contact with blood.

### BACKGROUND OF THE INVENTION

When blood samples are taken, for example, disposable micropipets in the form of capillary small tubes are used which are made of glass. The advantage of glass is that it can be coated in particular with heparin as a hemocompatible compound to a sufficient degree by the adsorption of heparin from an aqueous solution of heparin. Due to the high risk of breaking and the resulting risk of injuries and infections shown by the pipets, the material glass is replaced by breakage-proof plastics. However, the problem has occurred that such materials cannot be sufficiently coated with heparin in the usual manner in order to suppress the formation of thrombi or the coagulation of the blood for a sufficient period of time.

### SUMMARY OF THE INVENTION

It is the object of the present invention to provide a process for the anti-thrombogenic coating of artificial surfaces.

This object is solved by the technical teaching of the independent claims of the present invention. Further advantageous embodiments of the invention are evident from the dependent claims, the description and the examples.

### DETAILED DESCRIPTION OF THE INVENTION

The present invention discloses a process for the anti-thrombogenic coating of surfaces which are not blood-compatible, comprising the following steps:
a) contacting the surface with a solution of at least one amphiphilic compound,
b) drying the surface,
c) contacting the surface coated with amphiphilic compounds with a solution of at least one anticoagulant, and
d) finally, drying the anti-thrombogenic coated surface.

Surprisingly, it has been shown that all in particular hydrophobic surfaces can be coated in accordance with the inventive process, and the anti-thrombogenic coating protects the blood or the coagulable blood product for a sufficient time period of at least 24 hours from coagulation.

Such an anti-thrombogenic coating can be applied to various artificial materials such as metal, alloys, plastics, ceramics, minerals, glass or other materials. Metals and metal alloys such as medical high-quality steel LVM 316 and various plastics are preferred.

In particular, plastics such as polyethylene terephthalate, polyethylene, polypropylene, polyvinyl chloride, polyamide, polyurethane, polycarbonate, polysulfone, polyether etherketone, silicone, polytetrafluoroethylene, polystyrene, polymethyl methacrylate, polyvinylidene fluoride or mixtures or copolymers of the above-mentioned or other preferably hydrophobic plastics can be coated according to the invention. The process is preferably suitable for coating plastics with hydrophobic surfaces to which the hydrophilic anticoagulants adhere only insufficiently.

Surfaces coated this way are primarily used for medical products which come into direct contact with blood and coagulable blood products. These medical products include, for example, hypodermic needles, capillaries, valves, needles, containers, bags, conserves, catheters and tubes, in particular of plastics, glass, metal or metal alloys.

The process of the invention, in a first step, uses a solution of an amphiphilic compound, which is contacted with the surface to be treated. As the amphiphilic compounds, substances having at least one positively charged and/or at least positively polarized hydrophilic group and at least one hydrophobic group are used primarily. As the positively charged and/or positively polarized hydrophilic groups, ammonium, phosphonium and carbonium groups are suitable, and as the hydrophobic groups, long-chain alkyl groups, phenyl and benzyl groups are primarily considered, which can be present, for example, in quaternary ammonium or quaternary phosphonium salts and tertiary carbonium compounds such as triphenylmethyl cation.

The amphiphilic compound is preferably taken up in water or an organic solvent before it is contacted with the surface to be coated. As organic solvents, ethers such as dioxane, tetrahydrofuran (THF), petroleum ether, diethyl ether, methyl-Lert-butyl ether (MTBE), ketones such as acetone or propanone, alkohols such as methanol, ethanol, propanol, isopropanol, carboxylic acids such as formic acid, acetic acid, propionic acid, amides such as dimethylformamide (DMF) or dimethylacetamide, aromatic solvents such as toluene, benzene, xylene, pure hydrocarbon solvents such as pentane, hexane, cyclohexane and carbonic acid esters such as acetic acid ethyl ester are suitable. Mixtures of the above-mentioned solvents can also be used.

The solvent or solvent mixture is chosen depending on the type of the material to be coated, wherein it has to be taken into consideration that the material to be coated is not attacked or even dissolved by the solvent.

Mixtures of amphiphilic compounds can also be used according to the invention. In addition, the amphiphilic compound or the mixture of the amphiphilic compounds can be adapted adequately to the respective material to be coated.

The solution contains the amphiphilic compound or a mixture of the amphiphilic compounds in a concentration of 0.01 percent by weight to 50 percent by weight, preferably of 0.5 percent by weight to 10 percent by weight, and particularly preferably in a concentration of 1 percent by weight to 5 percent by weight.

As amphiphilic compounds, cationic surfactants, detergents, phase-transfer catalysts, emulgators and mixtures of said substances are used according to the invention.

Examples for cationic surfactants, detergents, phase-transfer catalysts or emulgators are tridodecylmethylammonium chloride (TDMAC), benzalkonium chloride (Fluka), ammonium salts such as tetrabutyl ammonium bromide, tetrabutyl ammonium fluoride, tributylmethyl ammonium bromide, tetrabutyl ammonium hydrogen sulfate, triethylbenzyl ammonium chloride, methyl tricapryl ammonium chloride; phosphonium salts such as triphenylethyl phosphonium acetate, triphenylethyl phosphonium bromide or iodide; Aliquat® HTA-1, Aliquat® 175, Aliquat® 128, Aliquat® 100 (all available from Cognis Aliquat); aromatic bisether imides such as 2,2-bis[4-(3,4-dicarboxy phenoxy)phenyl]propane-bis-N-methyl imide; diorgano aminopyridinium salts such as neopentyl dialkylaminopyridinium salts, N-2-ethyl hexyl dimethylaminopyridine chloride, N-2-ethyl hexyl-4-methylaminopiperidinyl aminopyridine chloride, N-2-ethyl hexyl dihexyl aminopyridine chloride; bisaminopyridinium salts such as tetraethylene glycol bis-(4-dimethylaminopyridinium)-bismethane sulfonate, 1,8-bis-(4-dimethylaminopyridinium)-octane dibromide, 1,10-bis-(4-dimethylaminopyridinium)-decane dibromide, 1,6-bis-(4-dihexyl aminopyridinium)-hexane dibromide.

The time of contact with the solution of the amphiphilic compound or the mixture of the amphiphilic compounds is at least 0.1 seconds, preferably between 0.1 and 60 seconds, and particularly preferably between 0.2 and 10 seconds.

In a second step, the solution of the amphiphilic compound(s) is removed, and the treated surface is dried. The second step of drying can be omitted in so far as, for the amphiphilic substance, a solvent or solvent mixture has been used which can be mixed with water or the solvent consisting of at least 80 percent by weight of water for the anticoagulant. Subsequently, the dried or aqueous wet surface is contacted with a solution of an anticoagulant.

According to the invention, as anticoagulants, substances are used which, in particular, are selected from the group comprising heparin, the salts thereof, salts of citric acid, salts of ethylenediaminetetraacetic acid, hirudin, sodium pentosan polysulfate, cumarin and derivatives thereof, warfarin, phenprocoumon and acenocoumarol. Preferably suitable anticoagulants are heparin, the salts thereof, salts of citric acid, salts of ethylenediaminetetraacetic acid and mixtures of said substances.

As the solvent for said anticoagulants, in particular, water or a solution having an aquosity of at least 80 percent by weight is used. As cosolvents, dioxane, tetrahydrofuran (THF), acetone, methanol, ethanol, propanol, isopropanol, acetic acid, dimethylformamide (DMF) or dimethylacetamide can be used.

The primarily aqueous solution contains the anticoagulant or mixtures of anticoagulants in a concentration of 0.01 percent by weight to 10 percent by weight, preferably of 0.05 percent by weight to 5 percent by weight, and particularly preferably in a concentration of 0.1 percent by weight to 1 percent by weight.

The time of contact with the solution of the anticoagulant or the mixture of anticoagulants is at least 0.1 seconds, preferably between 0.1 and 60 seconds, and particularly preferably between 0.2 and 10 seconds.

In a final step, the solution of the anticoagulant or the mixture of anticoagulants is removed, and the surface with the anti-thrombogenic coating is dried.

The surface coated according to the invention has a coverage density of anticoagulants which is at least sufficient for making the volume of the blood or the coagulable blood product coming into contact with the surface incoagulable. For heparin, this is 15 International Units (I.U.)/ml of blood. Using, for example, heparin from Serva with 196 I.U./mg (13,000 g/mol) and a capillary with a filling volume of 38 mm³ (188 mm² luminal surface of the capillary), the minimum coverage density required corresponds to: 38 mm³→0.57 I.U./capillary→0.303 I.U./cm² →1.55 μg/cm²→119 pmol/cm².

If, however, a system for taking blood samples designed for 9 ml of blood, a length of 5.5 cm, a diameter of 1.45 cm and an area of 36.3 cm² is taken as an example, the minimum coverage density required will correspond to: 9 ml→135 I.U./system for taking blood samples→135 I.U./36.3 cm²→3.72 I.U./cm²→0.019 mg/cm²→1.46 nmol/cm².

A further embodiment of the present invention concerns a process for the anti-thrombogenic coating of surfaces which are not blood-compatible, comprising the following steps:
a) contacting the surface with a solution of at least one amphiphilic compound and at least one anticoagulant, and
b) drying the surface.

In this process, first, a complex of the amphiphilic compound with the anticoagulant is prepared, and said solution of the complex is contacted with the surface to be treated. After a time of contact of at least 0.1 seconds, preferably between 0.1 and 60 seconds, and particularly preferably between 0.2 and 10 seconds, the solution is removed and the surface with the anti-thrombogenic coating is dried.

The amphiphilic compounds, anticoagulants, surfaces and solvents which are used correspond to those mentioned above. In all processes mentioned above, the coverage densities obtained can be adjusted within broad limits by changing the concentrations used of amphiphilic reagents and anticoagulants, so that a variety of medical products having various dimensions and shapes (volume/area ratios) are available for the process.

### EXAMPLES

### Example 1

A PET capillary was connected via an adapter to a hose, which was clamped into a hose pump. A solution of 44% of benzalkonium chloride from Fluka (60% of benzyl dimethyl dodecyl ammonium chloride, 40% of benzyl dimethyl tetradecyl ammonium chloride) in water was pumped through the capillary and out again. Subsequently, for drying, air was pumped through the capillary for 1.5 hours. Subsequently, a 0.25% solution of heparin was pumped through the capillary and out again. Then, drying was effected again by pumping air through.

The capillary coated this way was filled with blood. After 24 hours, the blood in the capillary was still free from thrombi.

4 further capillaries coated according to the method described above were cut into 4 pieces of the same length each and put into a hydrolysis tube. The tubes were charged with an amount of 3 M hydrochloric acid sufficient to adequately cover the fragments with liquid. An exactly dosed amount of heparin was also used in the hydrolysis. The hydrolysis tubes were closed tightly and hydrolyzed in a drying cabinet over night at 100° C. Subsequently, cooling was effected, the hydrolysis solution was flushed into a 100 ml flask without loss and the liquid evaporated with the help of a rotary evaporator at a water temperature of 50° C. Water was added once and evaporated again to dryness. The residues of the capillary hydrolysis remaining after the hydrochloric acid hydrolysis and evaporation were taken up in exactly 2 ml of distilled and filtered water. The heparin standard hydrolysis was taken up in 250 ml of water. These solutions were measured in an HPLC equipment with pulsed amperometric detection (BioQuant from Bischoff), and the measuring signals were evaluated quantitatively. HPLC eluent: 0.02 M NaOH, column: Carbopac PA1 from Dionex. A coverage density of 407 pmol/cm² was obtained, which is much higher than the required minimum coverage value, and thus effectively suppresses a coagulation of the blood or the coagulable blood product.

### Example 2

3.5 g of tridodecylmethylammonium chloride were dissolved in 100 ml of toluene/petroleum ether (1:1, v:v) and added to a solution of 2.25 g of sodium heparin in 50 ml of water. In a separating funnel, the two liquids were heavily agitated for one minute and allowed to separate over night.

The organic phase was pumped through a PET capillary and out again. Subsequently, air was pumped through the capillary for 1.5 hours for drying. The coverage density as specified in example 1 was 349 pmol/cm².

From the foregoing description, many different embodiments according to this invention will be possible. All such embodiments, including obvious variations of the particularly preferred processes disclosed herein, are intended to be within the scope of this invention, as defined by the claims that follow.

1. A process for the anti-thrombogenic coating of surfaces which are not blood-compatible, comprising the steps:
a) contacting the surface with a solution of at least one amphiphilic compound,
b) contacting the surface coated with at least one amphiphilic compound with a solution of at least one anticoagulant, and
c) drying the anti-thrombogenic coated surface.

2. A process for the anti-thrombogenic coating of surfaces which are not blood-compatible, comprising the steps:
a) contacting the surface with a solution of at least one amphiphilic compound,
b) drying the surface,
c) contacting the surface coated with amphiphilic compounds with a solution of at least one anticoagulant, and
d) finally, drying the anti-thrombogenic coated surface.

3. A process for the anti-thrombogenic coating of surfaces which are not blood-compatible, comprising the steps:
a) contacting the surface with a solution of at least one amphiphilic compound and at least one anticoagulant, and
b) drying the anti-thrombogenic coated surface.

4. The process according to any one of claims 1-3, wherein, as the amphiphilic compounds, substances having at least one positively charged and/or positively polarized hydrophilic group and at least one hydrophobic group are used.

5. The process according to claim 4, wherein, the amphiphilic compound is selected from cationic surfactants, detergents, phase-transfer catalysts and emulgators.

6. The process according to claim 5, wherein the amphiphilic compound is selected from the group comprising tridodecylmethylammonium chloride, benzalkonium chloride, quaternary ammonium salts, quaternary phosphonium salts, aromatic bisether imides, diorgano aminopyridinium salts and bisaminopyridinium salts.

7. The process according to any one of claims 1-3, wherein said surface is composed of metal, metal alloys, plastics, ceramics, minerals, and/or glass.

8. The process according to claim 7, wherein said surface is a plastic selected from the group comprising polyethylene terephthalate, polyethylene, polypropylene, polyvinyl chloride, polyamide, polyurethane, polycarbonate, polysulfone, polyether etherketone, silicone, polytetrafluoroethylene, polystyrene, polymethyl methacrylate, polyvinylidene fluoride and mixtures or copolymers of the above-mentioned plastics.

9. The process according to claim 7, wherein the surface is a hydrophobic surface.

10. The process according to any one of claims 1-3, wherein the solution of the at least one amphiphilic compound contains the amphiphilic compound in a concentration of 0.01 percent by weight to 50 percent by weight.

11. The process according to claim 10, wherein the solution of the at least one amphiphilic compound contains the amphiphilic compound in a concentration of 0.5 percent by weight to 10 percent by weight.

12. The process according to any one of claims 1-3, wherein said anticoagulant is selected from the group comprising heparin, salts of heparin, salts of citric acid, salts of ethylenediaminetetraacetic acid, hirudin, sodium pentosan polysulfate, cumarin, derivatives of cumarin, warfarin, phenprocoumon, acenocoumarol, and mixtures of said substances.

13. The process according to claim 12, wherein said anticoagulant is selected from heparin, salts of heparin, salts of citric acid, salts of ethylenediaminetetraacetic acid, and mixtures thereof.

14. The process according to any one of claims 1-3, wherein the solution of the at least one anticoagulant contains the anticoagulant in a concentration of 0.01 percent by weight to 10 percent by weight.

15. The process according to claim 14, wherein the solution of the at least one anticoagulant contains the anticoagulant in a concentration of 0.05 percent by weight to 5 percent by weight.

16. The process according to any one of claims 1-3, wherein the solution of the at least one anticoagulant has an aquosity of at least 80 percent by weight.

17. The process according to claim 1 or 2, wherein the time of contact of the surface with the solution of the at least one amphiphilic compound and with the solution of the at least one anticoagulant is at least 0.1 seconds.

18. The process according to claim 17, wherein said time of contact is between 0.2 and 10 seconds.

19. The process according to claim 3, wherein the time of contact of the surface with the solution of the at least one amphiphilic compound and the at least one anticoagulant is at least 0.1 seconds

20. The process according to claim 19, wherein the time of contact is between 0.2 and 10 seconds.

21. An anti-thrombogenic surface, prepared according to the process of any one of claims 1-3.

22. A medical product or device for direct contact with blood or coagulable blood products having an anti-thrombogenic surface according to claim 21.

23. The medical product or device of claim 22, wherein the medical product or device is selected from hypodermic needles, capillaries, valves, needles, containers, bags, conserves, catheters, and tubes, made of plastics, glass, metal or metal alloys.
